(19) United States
(12) Patent Application Publication
(10) Pub. No.: US 2023/0081529 A1
(43) Pub. Date: Mar. 16, 2023

(54) ANALYSIS METHOD FOR INCREASING SUSCEPTIBILITY TO SORAFENIB TREATMENT IN HEPATOCELLULAR CARCINOMA

(71) Applicant: CBSBIOSCIENCE CO., LTD, Daejeon (KR)

(72) Inventors: Jin-Young PARK, Seoul (KR); Hee-Jung WANG, Yongin-si, Gyeonggi-do (KR)

(73) Assignee: CBSBIOSCIENCE CO., LTD, Daejeon (KR)

(21) Appl. No.: 17/760,411

(22) PCT Filed: Feb. 1, 2021
(86) PCT No.: PCT/KR2021/001273

§ 371 Date: Aug. 9, 2022

(30) Foreign Application Priority Data
Feb. 12, 2020 (KR) 10-2020-0016737

(51) Int. Cl. C12Q 1/6886

(52) U.S. Cl. CPC C12Q 1/6886; C12Q 2600/106; C12Q 2600/158

(57) ABSTRACT

Provided is an analytical method of providing an information for diagnosis of a hepatocellular carcinoma patient having susceptibility to sorafenib, wherein the expression levels of eight genes, i.e., CDH1, CHAD, EFNA2, FANCC, MAP2K1, MEN1, PBRM1, and PPARG genes, in combination, are measured in carcinoma tissues of a hepatocellular carcinoma patient, and thereby the response (susceptibility) to sorafenib can be predicted with high accuracy, and the combination of said genes can be usefully applied as a biomarker for selecting a hepatocellular carcinoma patient having susceptibility to sorafenib.

### TECHNICAL FIELD

The present invention relates to an analytical method for increasing susceptibility of sorafenib therapy in hepatocellular carcinoma. More specifically, the present invention relates to an analytical method of providing an information for diagnosis of a hepatocellular carcinoma patient having susceptibility to sorafenib.

### BACKGROUND ART

Liver cancer is one of the major fatal cancers with high mortality and high incidence. In liver cancer cases, hepatocellular carcinoma (HCC) takes the major portions. During the therapeutic course, up to 50% of HCC patients received systemic therapy. In systemic therapy for HCC, sorafenib and lenvatinib were approved as first-line treatment without predictive biomarkers. However, sorafenib revealed 2% of objective response rates and 10.7 months of median overall survival (Llovet J M, et al., Sorafenib in advanced hepatocellular carcinoma. N Engl J Med 2008; 359: 378-390). This absence of predictive biomarkers caused non-selective treatment and showed poor response rates and overall survival. Also, most of agents for HCC lack predictive biomarkers. Many researchers have proposed that efforts to identify and validate new predictive biomarkers should be continued to be helpful to predict clinical efficacy and resistance to these agents (Califf R M. Biomarker definitions and their applications. Exp Biol Med (Maywood) 2018; 243:213-221; Twomey J D, Brahme N N, Zhang B. Drug-biomarker co-development in oncology—20 years and counting. Drug Resist Updat 2017; 30: 48-62).

Biomarkers are used for various purposes. Depending on the use, biomarkers are classified to a diagnostic biomarker, a predictive biomarker, a prognostic biomarker etc. Among them, a predictive biomarker has the function that it predicts a favorable or unfavorable effect for therapeutic intervention (Bhattacharyya A, Rai S N. Adaptive Signature Design review of the biomarker guided adaptive phase-III controlled design. Contemp Clin Trials Commun 2019; 15: 100378). In this therapeutic intervention degree, there are a disease control rate (DCR) which means rate of non-progression and an objective response rate (ORR) which means rate of traditional tumor response (Lara P N, Jr., et al., Disease control rate at 8 weeks predicts clinical benefit in advanced non-small-cell lung cancer results from Southwest Oncology Group randomized trials. J Clin Oncol 2008; 26: 463-467). The classification favorable group and unfavorable group for therapeutic intervention, through using predictive biomarkers, has important roles in clinical trials and clinical applications. Thus many researchers have been studied a predictive biomarker for various agents in most of diseases. The provision of right therapies to right patients is expected to be able to improve the quality of life by preventing unnecessary therapies.

### DISCLOSURE

### Technical Problem

The present inventors have performed various studies to develop clinically applicable biomarkers capable of predicting the disease control of sorafenib. Especially, the present inventors combined weighted genes to DCR gene signature and validated with various statistical analyses and meta-analyses. As a result, it has been found that, when analyzed in combination of the expression levels of specific genes, i.e., CDH1, CHAD, EFNA2, FANCC, MAP2K1, MEN1, PBRM1, and PPARG genes, the response (also referred to as “susceptibility”) to sorafenib treatment can be predicted with high accuracy.

Therefore, it is an object of the present invention to provide an analytical method for predicting a response to sorafenib treatment in a hepatocellular carcinoma patient, the method of which comprises using CDH1, CHAD, EFNA2, FANCC, MAP2K1, MEN1, PBRM1, and PPARG genes as biomarkers.

### Technical Solution

In accordance with an aspect of the present invention, there is provided an analytical method of providing an information for diagnosis of a hepatocellular carcinoma patient having susceptibility to sorafenib, the method of which comprises measuring expression levels of CDH1, CHAD, EFNA2, FANCC, MAP2K1, MEN1, PBRM1, and PPARG genes, in carcinoma tissue samples which are externally discharged from the hepatocellular carcinoma patient, respectively.

In the analytical method of the present invention, the measuring may be carried out by measuring the mRNA expression levels of CDH1, CHAD, EFNA2, FANCC, MAP2K1, MEN1, PBRM1, and PPARG genes, respectively.

### Advantageous Effects

It has been found by the present invention that, when analyzed in combination of the expression levels of specific genes, i.e., CDH1, CHAD, EFNA2, FANCC, MAP2K1, MEN1, PBRM1, and PPARG genes, in hepatocellular carcinoma tissues of a hepatocellular carcinoma patient, the response (susceptibility) to sorafenib treatment can be predicted with high accuracy. Therefore, the combination of said genes can be usefully applied as biomarkers for selecting a hepatocellular carcinoma patient having susceptibility to sorafenib.

### DESCRIPTION OF DRAWINGS

FIG. 1 shows a flowchart of gene signature development.

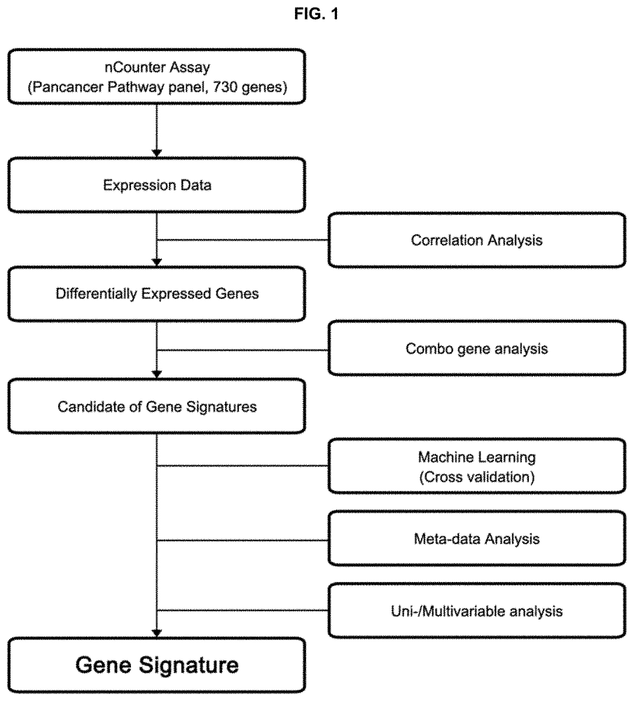

FIG. 2 shows the results obtained by evaluating clinical performance of the 8-gene signatures in predicting the disease control of sorafenib. FIG. 2A is the result of ROC (Receiver operating characteristic) analysis and FIG. 2B is the results of cross validation and logistic regression analyses.

FIG. 3 shows the results obtained by evaluating overall survival and progression free survival in predicted good responders versus predicted poor responders. FIG. 3A is the Kaplan-Meier curves for overall survival and FIG. 3B is the KM curves for progression free survival.

### BEST MODE FOR CARRYING OUT THE INVENTION

As used herein, the term “sorafenib” refers to the compound of the following Formula 1, including its pharmaceutically acceptable salt, for example p-toluenesulfonate salt.

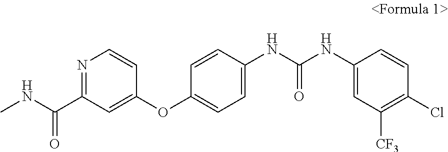 [SMILES: CNC(=O)c1cc(Oc2ccc(NC(=O)Nc3ccc(Cl)c(C(F)(F)F)c3)cc2)ccn1]

The term “patient having susceptibility to sorafenib” refers to a hepatocellular carcinoma patient showing response according to the sorafenib administration (i.e., tumor response). The “tumor response” refers to complete response, partial response, or stable disease according to the RECIST (Response Evaluation Criteria in Solid Tumors) defined in Llovet J M, et al. (2008) Sorafenib in advanced hepatocellular carcinoma. N Engl J Med 359: 378-390.

The term “hepatocellular carcinoma tissues” and “normal tissues” refer to the tissues samples externally discharged, via e.g., biopsy, from the hepatocellular carcinoma tissues and the surrounding normal tissues derived from a hepatocellular carcinoma patient. In clinics, carcinoma tissues and surrounding normal tissues are generally collected from a patient and then tissue examinations thereof are carried out for diagnosing hepatocellular carcinoma and/or establishing therapeutic regimen. Therefore, the term “hepatocellular carcinoma tissues” and “normal tissues” refer to the tissues samples externally discharged from a patient, e.g., for tissue examination in clinics.

Because of predictive biomarkers absence, sorafenib has poor response rates and overall survival period in hepatocellular carcinoma (HCC) therapy. The predictive biomarker could be a method that potentially improves the effectiveness of sorafenib. The present inventors have performed various studies to develop a clinically useful biomarker that predicts disease control of sorafenib. Using nCounter (Nanostring Technologies, Seattle, Wash.), the present inventors analyzed expression levels of 770 genes in 73 HCC patients who had received sorafenib treatment. As a result, we identified differentially expressed genes (DEGs) and computed combination of weighted gene expression for predictive biomarker. To validate gene signature, we analyzed cross validation and meta-analysis. As the results thereof, the 8-gene signature showed 0.90 of area under the curves (AUC), 91.78% of accuracy. In cross validation, the 8-gene signature showed 83.67% of cross validation accuracy. Also, the classification with the 8-gene signature revealed that median overall survival (median OS) was improved to 27.3 months from 11.3 months. Therefore, the 8-gene signature provides a best compromise between sorafenib effectiveness and coverage of sorafenib treatment patients.

Therefore, the present invention provides an analytical method of providing an information for diagnosis of a hepatocellular carcinoma patient having susceptibility to sorafenib, the method of which comprises measuring expression levels of CDH1, CHAD, EFNA2, FANCC, MAP2K1, MEN1, PBRM1, and PPARG genes, in carcinoma tissue samples which are externally discharged from the hepatocellular carcinoma patient, respectively.

In the analytical method of the present invention, the measuring may be carried out by measuring the mRNA expression levels of CDH1, CHAD, EFNA2, FANCC, MAP2K1, MEN1, PBRM1, and PPARG genes, respectively. The mRNA expression levels may be measured according to conventional methods used in the field of biotechnology, for example, by using nCounter PanCancer Pathway Panel (Nanostring Technologies, Seattle, Wash.).

In the analytical method of the present invention, the eight genes used as biomarkers, i.e., CDH1, CHAD, EFNA2, FANCC, MAP2K1, MEN1, PBRM1, and PPARG genes, are known in the art and the sequences thereof are known in GenBank and the like. For example, the NCBI Accession Numbers of the CDH1 (cadherin1) protein are NP_001304113, NP_001304114, NP_001304115, NP_004351 and the like; and the NCBI Accession Numbers of the genes encoding the same (mRNAs) are NM_004360, NM_001317184, NM_001317185, NM_001317186 and the like. The NCBI Accession Number of the CHAD (chondroadherin) protein is NP_001258; and the NCBI Accession Number of the gene encoding the same (mRNA) is NM_001267. The NCBI Accession Number of the EFNA2 (ephrin A2) protein is NP_001396; and the NCBI Accession Number of the gene encoding the same (mRNA) is NM_001405. The NCBI Accession Numbers of the FANCC (FA complementation group C) protein are NP_000127, NP_001230672 and the like; and the NCBI Accession Numbers of the genes encoding 3 the same (mRNAs) are NM_000136, NM_001243743 and the like. The NCBI Accession Number of the MAP2K1 (mitogen-activated protein kinase kinase 1) protein is NP_002746; and the NCBI Accession Number of the gene encoding the same (mRNA) is NM_002755. The NCBI Accession Numbers of the MEN1 (menin 1) protein are NP_000235, NP_570711, NP_570712, NP_570713, NP_570714 and the like; and the NCBI Accession Numbers of the genes encoding the same (mRNAs) are NM_000244, NM_130799, NM_130800, NM_130801, NM_130802 and the like. The NCBI Accession Numbers of the PBRM1 (polybromo 1) protein are NP_060783, NP_851385, NP_001337003, NP_001337004, NP_001337005 and the like; and the NCBI Accession Numbers of the genes encoding the same (mRNAs) are NM_018165, NM_018313, NM_181041, NM_181042, NM_001350074 and the like. The NCBI Accession Numbers of the PPARG (peroxisome proliferator activated receptor gamma) protein are NP_001317544, NP_005028, NP_056953, NP_619725, NP_619726 and the like; and the NCBI Accession Numbers of the genes encoding the same (mRNAs) are NM_005037, NM_015869, NM_138711, NM_138712, NM_001330615 and the like.

In an embodiment of the analytical method according to the present invention, after measuring the expression levels of CDH1, CHAD, EFNA2, FANCC, MAP2K1, MEN1, PBRM1, and PPARG genes in carcinoma tissue samples externally discharged from a hepatocellular carcinoma patient; when the TBPS (Treatment Benefit Prediction Score) value calculated according to the following equation is greater than −2.483069, the patient can be classified to a patient who exhibits a response to sorafenib treatment (i.e., a patient who exhibits susceptibility to sorafenib treatment) and when the TBPS value is −2.483069 or less, the patient can be classified to a patient who does not exhibit a response to sorafenib treatment (i.e., a patient who does not exhibit susceptibility to sorafenib treatment).

TBPS=(−0.000225)*G_{CDH1}+(0.001787)*G_{CHAD}+(−0.005687)*G_{EFNA2}+(−0.002104)*G_{FANCC}+(−0.001009)*G_{MAP2K1}+(0.002101)*G_{MEN1}+(−0.001336)*G_{PBRM1}+(0.001710)*G_{PPARG}

In the above equation, G_{CDH1}, G_{CHAD}, G_{EFNA2}, G_{FANCC}, G_{MAP2K1}, G_{MEN1}, G_{PBRM1}, and G_{PPARG} represent the gene expression levels of CDH1, CHAD, EFNA2, FANCC, MAP2K1, MEN1, PBRM1, and PPARG genes, respectively. That is, the expression level of each gene represents a normalized expression level obtained by using nCounter (Nanostring Technologies, Seattle, Wash.), a gene expression measuring device. The normalization is performed according to the manufacturers protocol, using nSolver Analysis Software v 3.0 (Nanostring Technologies), which is provided to nCounter (Nanostring Technologies, Seattle, Wash.).

The present invention will be described in further detail with reference to the following examples. These examples are for illustrative purposes only and are not intended to limit the scope of the present invention.

1. Test Methods

(1) Patients and Tissue Samples

This study included 73 histological confirmed HCC patients. HCC tissues were collected from the 73 patients before the sorafenib treatment. All tissues were obtained by needle biopsy. The patients were from Ajou Medical Center (AMC). The protocol of this study was approved by the Institutional Review Boards of Ajou Medical Center (AMC).

The HCC tissue samples were snap-frozen in liquid nitrogen and stored at −80° C. Complete clinical information was available for all cases. Patient staging information was obtained from CT or MRI images, and the Barcelona Clinic Liver Cancer (BCLC) staging was used.

(2) Measurement of Clinical Outcomes

Tumor response was assessed by computed tomography (CT) or magnetic resonance imaging (MRI) at 3 months and 6 months after administration of sorafenib based upon the modified Response Evaluation Criteria in Solid Tumors for HCC (mRECIST). From a DCR perspective, patients with complete response (CR), partial response (PR) and stable disease (SD) were considered as responders whereas those with progressive disease (PD) were judged as non-responders.

(3) RNA Extraction

Total RNA was extracted from both tumor and non-tumor tissues using the RNeasy mini kit (Qiagen, Hilden, Germany) with DNase I treatment (Qiagen, Hilden, Germany). Total RNA integrity was verified using a Bioanalyzer 2100 (Agilent Technologies, Santa Clara, Calif., USA). Total RNA concentration was measured using a Nanodrop 2000 (Thermo Fisher scientific, Waltham, Miss., USA).

(3) Gene Expression Assay

Gene expressions were analyzed by nCounter MAX (Nanostring, Technologies, Seattle, Wash., USA). Total reaction volume was 15 ul that contain 100 ng of RNA, reporter probes and capture probes. 770 genes (including 40 control genes) were analyzed through nCounter PanCancer Pathway Panel (Nanostring, Technologies, Seattle, Wash., USA). Quality control and normalization of the raw data was performed using nSolver Analysis Software v 4.0 (Nanostring Technologies, Technologies, Seattle, Wash., USA).

(4) Combination Gene Analysis

Differentially expressed genes (DEGs) were screened to meet one of the following conditions: 1) statistically significant difference comparing tumors with non-tumors or 2) statistically significant difference comparing sorafenib treatment responders with non-responders. The prior screened DEGs were further shortlisted through the logistic regression analysis for sorafenib response. Before combination of DEGs, we identified logistic regression coefficient of each genes and weighted gene expression with the corresponding coefficient value. The gene signature is calculated using the following equation:

Σ(correlation regression coefficient×gene expression value)

The number of shortlisted DEGs was analyzed in combination and the total number of gene combinations was calculated using the following equation:

$${\sum }_{k = 1}^{n} \frac{n !}{K ! ( n - k ) !}$$

In the above equation, n is the total number of shortlisted DEGs and k is the number of genes included in the combinations.

(5) Validation of candidate gene signatures by cross validation

The candidate gene signatures (p<0.05, AUC>0.08, sensitivity>85% and specificity>85%) were ranked by k-fold cross validation to identify the optimal gene combination. The patients were randomly separated by 2 folds (training set and test set), which were tested for 300 times repeatedly.

(6) Signal Transduction Pathway Analysis Based on Meta-Analysis

Signal transduction analysis was performed using meta-analysis program, CBS Probe PINGS™ (CbsBioscience, Daejeon, KOR) that consists of 5 modules (PPI module, Path-Finder module, Path-Linker module, Path-maker module and Path-Lister module). For gene signature validation, signal transduction was analyzed for pathway related with each patients DEGs compared tumor and non-tumor; and pathway related with gene signature. The genes were mapped to the signal transduction pathways obtained from KEGG (Kyoto Encyclopedia of Genes and Genomes database). We selected each top 10 signal transduction pathways for each patient's DEGs and gene signature according to the weight of numbers of interactions and interacting genes. And, we compared total pathway related with each patients DEGs and gene signature related pathway. Further we obtained interacting genes which mapped to the signal transduction pathways via KEGG (Kyoto Encyclopedia of Genes and Genomes database).

(7) Statistical Analysis

Relationship between treatment response and clinicopathologic variables was evaluated using Chi square tests or Fishers exact tests. Gene expression data were tested for normality with the Shapiro-Wilk test. As the total 73 data meet normality assumptions, significant differences between tumors and non-tumors were evaluated using the student t-test. As the data did not meet normality assumptions, significant differences between responders and non-responders were evaluated using the wilcoxon test. Receiver operating characteristic (ROC) curve analysis was used to determine accuracy of threshold values classifying tumor responders and non-responders using gene signature. Kaplan-Meier survival (KM) curves were calculated using death as end point in overall survival (OS) and using death and progression disease as end point in progression free survival (PFS). The difference in KM curves was examined by log-rank test and the difference in hazard ratio was examined by Cox regression analysis. Candidate Gene signatures were analyzed using logistic regression to measure the relationships between response to sorafenib treatment, classification, and clinicopathologic variables. Significance was set at P<0.05 (two-tailed). All statistics were performed in R version 3.3.3 (R Development Core Team, https:/www.r-project.org/).

2. Test Results

(1) Clinicopathologic characteristics of patients

In 73 patients treated with sorafenib, responders were 21 and non-responders were 52. There were no statistical differences between responders and non-responders in gender, HBV, HCV, TMN stage, and BCLC stage. However, in responder groups, patients whom age was over than 55 years were large portions. Patients whom AFP (alpha fetoprotein) was under 100 ng/ml were large portions in responders group, reverse to non-responders group (Table 1).

TABLE 1

| Clinicopathologic | Responders | Non-responders | |
|---|---|---|---|
| parameters | (n = 21) | (n = 52) | p-value* |
| Age (range) | | | |
| <55years | 3 (14.3%) | 23 (44.2%) | 0.0317 |
| ≥55years | 18 (85.7%) | 29 (55.8%) | |
| Gender (M/F) | | | |
| Male | 16 (76.2%) | 41 (78.8%) | 0.7657 |
| Female | 5 (23.8%) | 11 (21.2%) | |
| HBV (hepatitis B virus) | (−1) | | |
| Absent | 3 (15.0%) | 7 (13.5%) | 1 |
| Present | 17 (85.0%) | 45 (86.5%) | |
| HCV (hepatitis C virus) | (−1) | | |
| Absent | 20 (100.0%) | 49 (94.2%) | 0.5553 |
| Present | 0 (0.0%) | 3 (5.8%) | |
| BCLC stage | | | |
| A | 1 (4.8%) | 1 (1.9%) | 0.3493 |
| B | 2 (9.5%) | 13 (25.0%) | |
| C | 18 (85.7%) | 37 (71.2%) | |
| D | 0 (0.0%) | 1 (1.9%) | |
| AFP | (−1) | (−7) | |
| <100 ng/ml | 13 (65.0%) | 15 (33.3%) | 0.0350 |
| ≥100 ng/ml | 7 (35.0%) | 30 (66.7%) | |
| Tumor response | | | |
| Complete response | 2 (9.5%) | 0 (0.0%) | |
| Partial response | 9 (42.9%) | 0 (0.0%) | |
| Stable disease | 10 (47.6%) | 0 (0.0%) | |
| Progressive disease | 0 (0.0%) | 52 (100.0%) | |

*p values were calcuated using the Fisher's exact test.

(2) Differential Expression Gene Analyses of Tumors Versus Non-Tumors, and Responders Versus Non-Responders

DEG analysis between tumors and non-tumors revealed 507 out of 730 genes were significantly differentially expressed between tumors and non-tumors. And, DEG analysis between responders and non-responders to sorafenib treatment revealed 49 out of 730 genes were significantly differentially expressed between responders and non-responders to sorafenib treatment. The total numbers of genes that meet screening conditions were 525 genes (including 31 overlapping genes). When logistic regression analysis was performed with prior screened genes, the analysis revealed 26 DEGs were statistically significant (Table 2).

TABLE 2
Sorafenib response correlated genes in DEGs

| | | | | | | | | Screen condition |
|---|---|---|---|---|---|---|---|---|
| | | Logistic | Logistic | Screen condition 1 | | | | Responder vs |
| | | regression | regression | T mean | NT mean | Fold | T vs NT | Non-responder |
| No. | Gene | coefficient | p-value | (n = 81) | (n = 51) | change | p-value* | p-value¶ |
| 1 | AR | | 3.58E−02 | 1707.42 | 5590.40 | −3.51 | 2.20E−16 | — |
| 2 | CD14 | | 1.91E−02 | 4686.37 | 15061.60 | −3.21 | 1.94E−12 | — |
| 3 | CDC148 | | 5.68E−03 | 721.22 | 1583.62 | −2.20 | 1.88E−15 | — |
| 4 | CDH1 | −0.000225 | 1.92E−02 | 5253.03 | 8897.98 | −1.71 | 7.36E−10 | 2.09E−02 |
| 5 | CHAD | 0.001787 | 2.19E−02 | 354.29 | 752.81 | −2.12 | 4.69E−08 | — |
| 6 | EFNA2 | −0.005687 | 1.53E−02 | 212.88 | 297.39 | −1.4 | 4.76E−03 | 1.46E−02 |
| 7 | FANCC | −0.002104 | 4.15E−02 | 714.64 | 529.24 | 1.35 | 4.13E−05 | 3.66E−02 |
| 8 | FANCL | | 9.24E−03 | 724.58 | 820.53 | −1.13 | 1.66E−02 | 9.28E−03 |
| 9 | IL1R1 | | 3.70E−02 | 769.27 | 1708.03 | −2.22 | 3.11E−10 | — |
| 10 | KAT2B | | 3.33E−02 | 1263.95 | 1911.99 | −1.51 | 9.41E−09 | — |
| 11 | LIG4 | | 4.80E−02 | 698.01 | 1003.63 | −1.44 | 2.96E−09 | — |
| 12 | MAP2K1 | −0.001009 | 3.65E−02 | 1984.00 | 5323.86 | −2.68 | 6.14E−15 | 2.46E−02 |
| 13 | PBRM1 | −0.001336 | 3.41E−02 | 1664.14 | 1838.44 | −1.1 | 1.03E−02 | 6.22E−03 |
| 14 | PIK3CB | | 3.43E−02 | 684.01 | 610.41 | 1.12 | 2.51E−02 | 2.78E−02 |
| 15 | PPARG | 0.001710 | 1.71E−02 | 674.21 | 508.69 | 1.33 | 5.67E−04 | 3.05E−02 |
| 16 | PPP2R1A | | 4.38E−02 | 5679.31 | 4752.90 | 1.19 | 5.29E−04 | — |
| 17 | PRKCA | | 4.41E−02 | 852.48 | 633.07 | 1.35 | 1.29E−04 | — |
| 18 | RAD21 | | 4.47E−02 | 6629.89 | 4231.07 | 1.57 | 1.93E−08 | 1.51E−02 |
| 19 | RFC4 | | 4.74E−02 | 1269.50 | 373.64 | 3.4 | 4.60E−16 | 1.78E−02 |
| 20 | SOCS1 | | 3.35E−02 | 284.01 | 589.12 | −2.07 | 3.29E−03 | 1.15E−02 |
| 21 | TNFSF10 | | 1.74E−02 | 4217.80 | 8154.5 | −1.93 | 6.14E−09 | 3.45E−02 |
| 22 | ACVR1B | | 2.76E−02 | — | — | — | — | 3.00E−03 |
| 23 | FGF2 | | 4.53E−02 | — | — | — | — | 8.33E−03 |
| 24 | GTF2H3 | | 3.67E−02 | — | — | — | — | 1.41E−02 |
| 25 | MEN1 | 0.002101 | 2.10E−02 | — | — | — | — | 3.55E−02 |
| 26 | POLB | | 3.97E−02 | — | — | — | — | 4.37E−02 |

Data that do not meet screening conditions were not shown. Logistic regression coefficients of genes composing 8-gene signature were shown.

* p values were calculated using the student t-test.

¶ p values were calculated using the wilcoxon test.

(3) Gene Combination Analysis and Candidate Gene Signatures

Top 5 candidate gene signatures were ranked with AUC, and their AUC, sensitivity, and specificity are shown in Table 3 below.

TABLE 3

| | | | No. of | | | |
|---|---|---|---|---|---|---|
| | | Logisitic | Combi- | | | |
| | | Regression | nation | | Sensi- | Speci- |
| Rank | Gene Signature | p-value | genes | AUC | tivity | ficity |
| 1 | CD14_CDH1_EFNA2_LIG4_MEN1_PBRM1_POLB_PPARG_TNFSF10 | 1.40E−07 | 9 | 0.920 | 85.71 | 92.31 |
| 2 | AR_CDH1_EFNA2_FANCC_MAP2K1_MEN1_PBRM1 _PPARG | 1.40E−07 | 8 | 0.906 | 85.71 | 92.31 |
| 3 | CDH1_EFNA2_FANCC_MEN1_PBRM1_PPARG_RFC4_SOCS1 | 1.80E−07 | 8 | 0.902 | 90.48 | 92.31 |
| 4 | CDH1_CHAD_EFNA2_FANCC_MAP2K1_MEN1_PBRM1_PPARG | 1.03E−07 | 8 | 0.895 | 85.71 | 94.23 |
| 5 | CDH1_EFNA2_FANCC_KAT2B_MAP2K1_MEN1_PBRM1_PPARG | 1.03E−07 | 8 | 0.888 | 85.71 | 94.23 |

(4) Gene Signature Selection with Cross Validation

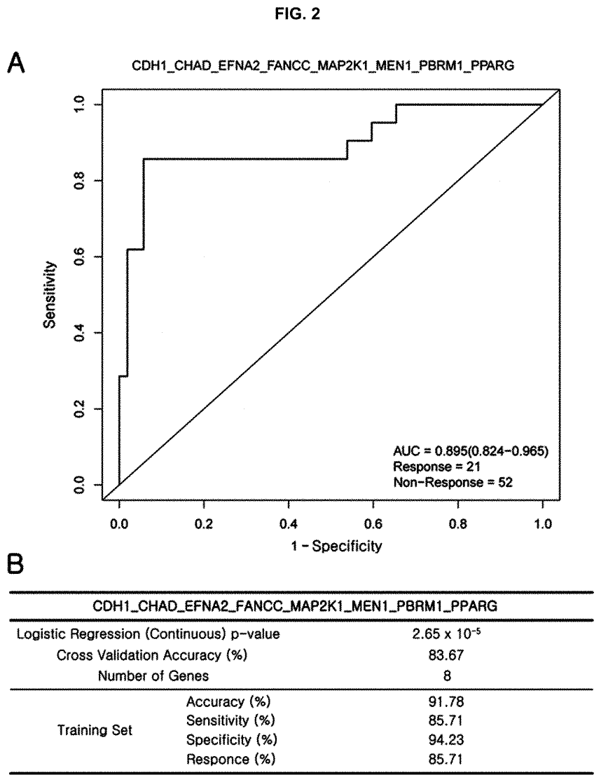

Top 5 candidate gene signatures were validated with cross validation. Rank 1 gene signature (CD14_CDH1_EFNA2_LIG4_MEN1_PBRM1_POLB_PPARG_TNFSF10) showed cross validation accuracy=82.00. Rank 2 gene signature (AR_CDH1_EFNA2_FANCC_MAP2K1_MEN1_PBRM1_PPARG) showed cross validation accuracy=82.00. Rank 3 gene signature (CDH1_EFNA2_FANCC_MEN1_PBRM1_PPARG_RFC4_SOCS1) showed cross validation accuracy=80.33. Rank 4 gene signature (CDH1_CHAD_EFNA2_FANCC_MAP2K1_MEN1_PBRM1_PPARG) showed cross validation accuracy=83.67 (FIG. 2). Rank 5 gene signature (CDH1_EFNA2_FANCC_KAT2B_MAP2K1_MEN1_PBRM1_PPARG) showed cross validation accuracy=81.33. With the cross validation accuracy, we selected Rank 4 gene signature (CDH1_CHAD_EFNA2_FANCC_MAP2K1_MEN1_PBRM1_PPARG) as gene signature for predicting sorafenib response.

(5) Calculation of Treatment Benefit Prediction Score (TBPS) through Logistic Regression Analysis

The regression coefficient values for each gene obtained through the univariate logistic regression analysis on the combination of the eight genes selected by the above method (i.e., CDH1, CHAD, EFNA2, FANCC, MAP2K1, MEN1, PBRM1, and PPARG) are shown in the following Table 4.

TABLE 4

| Gene | Regression coefficient value |
|---|---|
| CDH1 | −0.000225 |
| CHAD | 0.001787 |
| EFNA2 | −0.005687 |
| FANCC | −0.002104 |
| MAP2K1 | −0.001009 |
| MEN1 | 0.002101 |
| PBRM1 | −0.001336 |
| PPARG | 0.001710 |

A Treatment Benefit Prediction Score (TBPS) was calculated according to the following equation, using the normalized expression levels of the respective 8 genes (i.e., CDH1, CHAD, EFNA2, FANCC, MAP2K1, MEN1, PBRM1, and PPARG) obtained with nCounter (Nanostring Technologies, Seattle, Wash.) and the regression coefficient values for each gene.

TBPS=C_{CDH1}*G_{CDH1}+C_{CHAD}*G_{CHAD}+C_{EFNA2}*G_{EFNA2}+C_{FANCC}*G_{FANCC}+C_{MAP2K1}*G_{MAP2K1}+C_{MEN1}*G_{MEN1}+C_{PBRM1}*G_{PBRM1}+C_{PPARG}*G_{PPARG}

In the above equation, C_{gene} represents the regression coefficient value of the corresponding gene; and G_{gene} represents the normalized expression level of the corresponding gene which was obtained with nCounter (Nanostring Technologies, Seattle, Wash.). Thus, from the results of Table 4, the TBPS can be also calculated according to the following equation.

TBPS=(−0.000225)*G_{CDH1}+(0.001787)*G_{CHAD}+(−0.005687)*G_{EFNA2}+(−0.002104)*G_{FANCC}+(−0.001009)*G_{MAP2K1}+(0.002101)*G_{MEN1}+(−0.001336)*G_{PBRM1}+(0.001710)*G_{PPARG}

The calculated TBPS value as described above is −2.483069, which can be used as a threshold capable of predicting a response to sorafenib therapy. That is, after measuring the expression levels of CDH1, CHAD, EFNA2, FANCC, MAP2K1, MEN1, PBRM1, and PPARG genes in a hepatocellular carcinoma patient; when the TBPS value calculated according to the above equation is greater than −2.483069, the patient can be classified to a patient who exhibits a response to sorafenib treatment (i.e., a patient who exhibits susceptibility to sorafenib treatment) and when the TBPS value is −2.483069 or less, the patient can be classified to a patient who does not exhibit a response to sorafenib treatment (i.e., a patient who does not exhibit susceptibility to sorafenib treatment).

(6) Kaplan-Meier Analysis about Prognosis Between 8-Gene Signature High Group and Low Group

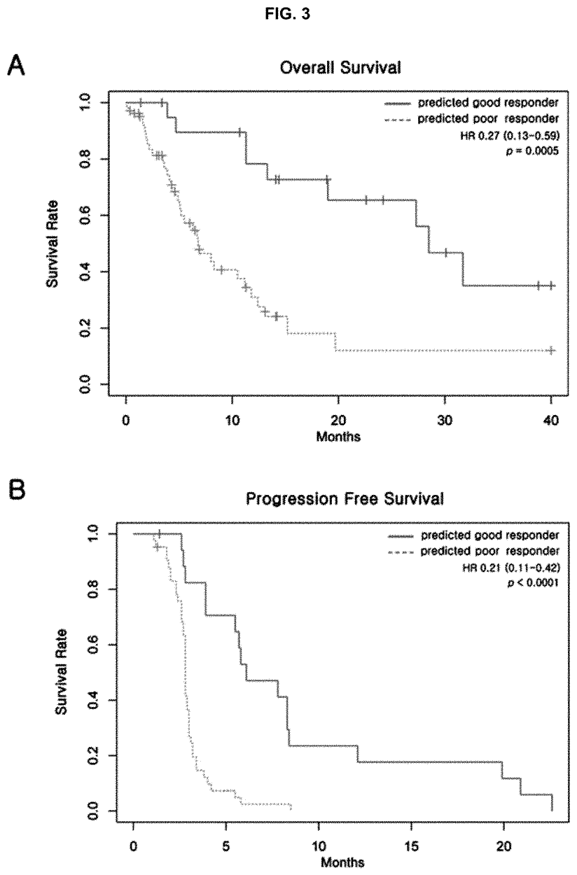

Using the KM analysis, prognosis between predicted good responders and predicted poor responders was investigated in overall survival and progression free survival. In overall survival, median overall survival of entire patients was 11.3 months (95% CI; 4.6-11.2), median overall survival of predicted good responders was 27.3 months (95% CI; 11.3-28.5) and median overall survival of predicted poor responders was 6.7 months (95% CI; 3.6-6.8). Hazard ratio between predicted good responders and predicted poor responders was 0.27 (95% CI; 0.13-0.59, p-value=0.0005). In progression free survival, median survival of entire patients was 2.9 months (95% CI; 2.8-3.4), median survival of predicted good responders was 5.8 months (95% CI; 3.9-8.4) and median survival of predicted poor responders was 2.8 months (95% CI; 2.7-3.0). Hazard ratio between predicted good responders and predicted poor responders was 0.21 (95% CI; 0.11-0.42, p-value<0.0001). In time to progression, median period of entire patients was 2.9 months (95% CI; 2.8-3.4), median period of predicted good responders was 5.8 months (95% CI; 3.9-8.4) and median period of predicted poor responders was 2.8 months (95% CI; 2.7-3.0). Hazard ratio between predicted good responders and predicted poor responders was 0.22 (95% CI; 0.11-0.44, p-value<0.0001) (FIG. 3).

TABLE 5

| | | Predicted good | Predicted poor |
|---|---|---|---|
| | | responders to | responders to |
| | Sorafenib | sorafenib | sorafenib |
| | treatment | treatment | treatment |
| Median | 11.3 months | 27.3 months | 6.7 months |
| overall survival | (4.6-11.2 months) | (11.3-28.5 months) | (3.6-6.8 months) |
| Median | 2.9 months | 5.8 months | 2.8 months |
| progression-free | (2.8-3.4 months) | (3.9-8.4 months) | (2.7-3.0 months) |
| survival | | | |
| Median | 2.9 months | 5.8 months | 2.8 months |
| time to | (2.8-3.4 months) | (3.9-8.4 months) | (2.7-3.0 months) |
| progression | | | |

(7) Logistic Regression Analysis about Independency of Selected Gene Signature

The independency of selected gene signature (CDH1_CHAD_EFNA2_FANCC_MAP2K1_MEN1_PBRM1_PPARG) was investigated with logistic regression analysis. In univariable logistic regression analysis, age and gene signature were significantly different and positively correlated with sorafenib response. However, AFP was significantly different and negatively correlated with sorafenib response. In multivariable logistic analysis with significantly correlated factor with sorafenib response, AFP was not showed independency with other factors, but age and gene signature were independent predictors of sorafenib response (Tables 6 and 7).

TABLE 6
Univariable logistic regression analysis

| | | | | | |
|---|---|---|---|---|---|
| | | | se(co- | | |
| variable | n | coefficient | efficient) | z | p-valiue |

Candidate genes

| | | | | | |
|---|---|---|---|---|---|
| CDH1_CHAD_EFNA2_FANCC_MAP2K1_MEN1_PBRM1_PPARG | 73 | 4.5850 | 0.8618 | 5.321 | 1.03E−07 |
| (low vs high) | | | | | |

Clinicopathological feature

| | | | | | |
|---|---|---|---|---|---|
| Age (<55 years vs ≥55 years) | 73 | 1.5600 | 0.6833 | 2.283 | 0.0224 |
| Gender (male vs female) | 73 | 0.1525 | 0.6147 | 0.248 | 0.8040 |
| HBV (absent vs present) | 72 | −0.1262 | 0.7465 | −0.169 | 0.8660 |
| HCV (absent vs present) | 72 | −15.6700 | 1385.3778 | −0.011 | 0.9910 |
| TNM stage (I-II vs III-IV) | 73 | −1.4271 | 0.9534 | −1.497 | 0.1340 |
| BCLC (A-B vs C-D) | 73 | 0.7932 | 0.6976 | 1.137 | 0.2555 |
| AFP (<100 ng/ml vs ≥100 ng/ml) | 65 | −1.3122 | 0.5655 | −2.320 | 0.0203 |

TABLE 7
Multivariable logistic regression analysis

| variable | Odds ratio | 95% Cl | p-value |
|---|---|---|---|
| CDH1_CHAD_EFNA2_FANCC_MAP2K1_MEN1_PBRM1_PPARG | 139.90 | 12.72-1538.21 | 5.36E-05 |
| (low vs high) | | | |
| Age (<55 years vs ≥55 years) | 13.60 | 1.17-157.62 | 0.0368 |
| AFP (<100 ng/ml vs ≥100 ng/ml) | 1.05 | 0.15-7.31 | 0.9625 |

(8) Signal Transduction Pathway Analysis and High Interaction Frequency Ratio Genes Analysis for Selected Gene Signature

Signal transduction analysis through the meta-analysis showed that the gene signature of sorafenib responders was related highly to the 6 pathways, i.e., pathways in cancer, human papillomavirus infection, proteoglycans in cancer, P13K-Akt signaling pathway, focal adhesion, and Ras signaling pathway. And, 3 genes (EGFR, CTNNB1, SRC) were high interaction frequency ratio genes related with both gene signature and sorafenib responders (Table 8).

TABLE 8
Gene signature related pathways and high
interaction frequency ratio genes

| | Name of pathway/High interaction |
|---|---|
| | frequency ratio genes |
| Pathway | Pathways in cancer |
| | Human papillomavirus infection |
| | Proteoglycans in cancer |
| | PI3K-Akt signaling pathway |
| | Focal adhesion |
| | Ras signaling pathway |
| High interaction frequency | EGFR |
| ratio genes | CTNNB1 |
| | SRC |

3. Discussion

The present inventors investigated mRNA expression of 730 genes with nCounter system in HCC tumors tissues and surrounding non-cancerous tissues, and then identified 525 DEGs and 26 genes correlated with disease control response of sorafenib. To apply influence of various genes to response, we analyzed logistic regression coefficient of each genes and weighted to corresponding gene expression values. Based on weighted gene expression value, the present inventors computed all of gene combination and validated candidate gene signature with cross validation. Kaplan-Meier analysis, meta-analysis and uni-variable/multi-variable analysis were conducted.

With cross validation performance, we selected CDH1_CHAD_EFNA2_FANCC_MAP2K1_MEN1_PBRM1_PPARG of 8-gene signature. The 8-gene signature achieved 91.78% accuracy and 83.67% cross validation accuracy with −2.483069 of cutoff. Meta-analysis revealed that EGFR and CTNNB1, SRC were interacting with 8 genes composing gene signature. In this result, EGFR confers resistance to sorafenib through Akt activation and SRC confers resistance to sorafenib through FAK-SRC signaling pathway as bypass track (Ezzoukhry Z, et al., EGFR activation is a potential determinant of primary resistance of hepatocellular carcinoma cells to sorafenib. Int J Cancer 2012; 131: 2961-2969; Zhou Q, et al., Activation of Focal Adhesion Kinase and Src Mediates Acquired Sorafenib Resistance in A549 Human Lung Adenocarcinoma Xenografts. J Pharmacol Exp Ther 2017; 363: 428-443). Additionally, P13K-Akt signaling pathway and Focal adhesion pathway were observed interacting with the 8-gene signature. With this biological explanation supported, the 8-gene signature increased disease control rate from 28.77% to 85.71%. In predicted good responders, prognoses of OS and PFS showed improvement than entire patients and predicted poor responders.

This 8-gene signature might be the best compromise between sorafenib effectiveness and coverage of sorafenib treatment patients, because sorafenib has low overall response rate. Therefore, the 8-gene signature can be usefully applied as a DCR biomarker for predicting the response to sorafenib in HCC patients.

1. An analytical method of providing an information for diagnosis of a hepatocellular carcinoma patient having susceptibility to sorafenib, the method of which comprises measuring expression levels of CDH1, CHAD, EFNA2, FANCC, MAP2K1, MEN1, PBRM1, and PPARG genes, in carcinoma tissue samples which are externally discharged from the hepatocellular carcinoma patient, respectively.

2. The analytical method according to claim 1, wherein the measuring is carried out by measuring the mRNA expression levels of CDH1, CHAD, EFNA2, FANCC, MAP2K1, MEN1, PBRM1, and PPARG genes, respectively.
